(19) United States
(12) Patent Application Publication
(10) Pub. No.: US 2025/0087345 A1
(43) Pub. Date: Mar. 13, 2025

(54) MEDICAL DEVICE REPAIR SUPPORT

(71) Applicant: Patricia M. Currie, St. Andrews (AU)

(72) Inventor: Patricia M. Currie, St. Andrews (AU)

(21) Appl. No.: 18/894,685

(22) Filed: Sep. 24, 2024

Related U.S. Application Data
(63) Continuation of application No. PCT/US2023/016130, filed on Mar. 23, 2023.
(60) Provisional application No. 63/269,853, filed on Mar. 24, 2022.

(51) Int. Cl. G16H 40/40

(52) U.S. Cl. CPC G16H 40/40

(57) ABSTRACT

Various examples are directed to systems and methods for servicing a medical device comprising an onboard memory device. An interface computing system may establish a communication link with the medical device. Usage history data describing the medical device may be obtained via the communication link. Service history data describing at least one repair for the medical device may be obtained. Age data describing an age of the medical device may be obtained. Status data for the medical device may be determined using the usage history data, the age data, and the service history data. A medical device servicer system may initiate a service activity for the medical device based at least in part on the status data.

### PRIORITY CLAIM

This application is a Continuation of International Patent Application No. PCT/US2023/016130, filed Mar. 23, 2023, which claimed the benefit of priority to U.S. Provisional Patent Application Ser. No. 63/269,853, filed Mar. 24, 2022, the benefit of each of which is hereby presently claimed, and the entireties of each are hereby incorporated by reference herein.

### BACKGROUND

Medical devices can be used for various different purposes. Endoscopes are one example type of medical device. Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

### BRIEF DESCRIPTION OF DRAWINGS

The present disclosure is illustrated by way of example and not limitation in the following figures.

FIG. 1 is a diagram showing one example of an environment for supporting medical device repair.

FIG. 2 is a flowchart showing one example of a process flow that may be executed to determine whether a medical device is eligible for service.

FIG. 3 is a flowchart showing one example of a process flow that may be executed by the medical device servicer system of FIG. 1 to determine whether the medical device is eligible for service.

FIG. 4 is a diagram showing one example of a user interface screen that may be served to a user computing device associated with a medical device consumer user.

FIG. 5 is a flowchart showing one example of a process flow that may be executed, for example, by the medical device servicer system of FIG. 1, to determine status data for a medical device.

FIG. 6 is a diagram showing one example of an environment including an example medical device in communication with an interface computing device, such as a reader or a user computing device.

FIG. 7 is a schematic diagram of one example of an endoscopy system comprising imaging and control system and endoscope.

FIG. 8 is a schematic diagram of one example of the endoscopy system of FIG. 7 comprising imaging and control system and endoscope.

FIG. 9 is a flowchart showing one example of a process flow that may be executed to determine a status of a medical device.

FIG. 10 is a diagram showing one example of an environment including the medical device servicer system in communication with various other components.

FIG. 11 is a flowchart showing one example of a process flow that may be executed in the environment of FIG. 10 to facilitate servicing of the medical device.

FIG. 12 is a flowchart showing another example of a process flow that may be executed, for example, in the environment of FIG. 10.

FIG. 13 is a flowchart showing another example of a process flow that may be executed, for example, in the environment of FIG. 10.

FIG. 14 is a block diagram showing one example of a software architecture for a computing device.

FIG. 15 is a block diagram of a machine in the example form of a computer system within which instructions may be executed for causing the machine to perform any one or more of the methodologies discussed herein.

### DESCRIPTION

Various examples described herein are directed to systems and methods for servicing medical devices, such as, for example endoscopes. Medical devices, such as endoscopes, may be produced by a manufacturer and provided to a medical device consumer. A medical device consumer may be a person or entity that uses one or more medical devices. Examples of medical device consumers may include hospitals, physician's offices, outpatient facilities, and/or the like. Medical device consumers may be and/or be associated with one or more practitioners who may utilize medical devices to treat patients. The medical device consumer may purchase, rent, or otherwise acquire medical devices for use by the practitioners to treat patients.

Some medical devices, such as endoscopes, are designed for repeated use. For example, a medical device consumer (e.g., associated practitioners thereof) may utilize a medical device multiple times for multiple procedures. The medical device may be cleaned and sterilized between procedures, either directly by the medical device consumer or using a third-party.

The lifecycle of many reusable medical devices includes periodic repair. For example, if a medical device breaks and/or its capabilities are degraded, it may be desirable to repair the medical device before using it for additional procedures. Many medical device consumers lack the facilities and/or expertise to repair medical devices such as endoscopes. Accordingly, a medical device consumer may utilize a medical device servicer to perform repairs on medical devices. In some examples, the medical device servicer for various medical devices is the enterprise that originally manufactured the medical devices, although in some examples the medical device servicer may be a third-party.

In various examples, reusable medical devices have a finite suggested usage life. At the expiration of the suggested usage life of a medical device, the medical device consumer may retire the medical device and, if desirable, replace the medical device with a new medical device.

In some examples, it may be challenging to determine when a medical device has reached the end of it suggested usage life. For example, some medical devices are used more frequently than others. A medical device that is used, for example, for two thousand procedures per year may reach the end of its suggested usage life faster than a similar medical device that is used for one thousand procedures per year. Also, the wear and tear on a medical device may depend on factors other than the raw number of procedures performed using the device. For example, some medical devices may require repair, including major repair, more frequently than other similar medical devices. Medical devices that require repair, including major repair, more frequently may reach the end of their suggested usage life faster than other medical devices.

One of the reasons that it may be challenging to determine when a medical device has reached the end of its suggested usage life is that use of the medical device and the repair function for the medical device may be distributed among different parties. For example, as described herein, the medical device may be used to perform procedures at a facility of the medical device consumer and repaired at a facility of a medical device servicer. Accordingly, if information about the usage of the medical device is tracked, that tracking may be performed at the facility of the medical device consumer and may not be readily available to the medical device servicer. On the other hand, as described herein, repairs of the medical device may occur off-site from the medical device consumer at the facility of a medical device servicer. Accordingly, it is the medical device servicer that may have data regarding repairs performed to a medical device, and such data may not be readily available to the medical device consumer.

In various examples, medical device consumers and servicers may address the challenges of determining the suggested end of usage life for a medical device by utilizing an age threshold. For example, the suggested usage life for some endoscope medical devices may be six years. Such an age threshold may be applied to all medical devices, regardless of usage or repair history. In this way, complexities regarding pooling of information to determine a more precise end of suggested usage life is avoided.

Utilizing an age threshold for retiring or replacing medical devices, however, presents its own disadvantages. Some medical devices may remain suitable for use even after reaching an age threshold. For example, if a medical device is used for fewer procedures and/or has necessitated major repairs less often, it may have a longer effective usage life than other similar medical devices. When an age threshold is used, for example, such medical devices may be scrapped even though they may be suitable for further use, thus wasting a portion of their usable life.

Various examples address these and other challenges utilizing various components, including medical devices, an interface computing device, and a medical device servicer system. The medical device may be configured to include an onboard memory. At the onboard memory, the medical device may store data including usage history data describing procedures that have been performed using the medical device. The usage history data may include, for example, a number of procedures performed. In some examples, the usage history data may also include information about each procedure performed using the medical device including, for example, a procedure type, a procedure duration, and/or the like. The medical device may additionally include a communication circuit that may be used to establish a communication link between the medical device and the interface computing device. The usage history data may be provided to the interface computing device via the communication link.

The interface computing device may be in communication with a medical device servicer system and may communicate the usage history data to the medical device servicer system. The medical device servicer system may utilize the usage history data (e.g., and other data) to determine status data describing a status of the medical device. The status data may describe a status of the medical device relative to an end of the suggested usage life. For example, the status data may indicate that the medical device is suitable for continued use. In other examples, status data may indicate that the medical device is unsuitable for continued use and should be replaced. Other possible statuses may also be found. For example, some medical devices may have a status indicating that the device is suitable for continued use, but should not be repaired if a (further) repair is needed.

Utilizing the example medical devices, interface computing devices, and medical device servicer systems described herein may improve the operation of a medical device. The example medical devices, interface computing devices, and medical device servicer systems described herein may safely lengthen the usable life of some medical devices. For example, some medical devices that may have previously been scrapped at the expiration of an age threshold may enjoy continued safe usage. It will be appreciated that, for example, utilizing usage history data stored at the onboard memory of the medical device in conjunction with other relevant data accessible to the medical device servicer system may provide for finer determinations of when the medical device is or is not suitable for continued use.

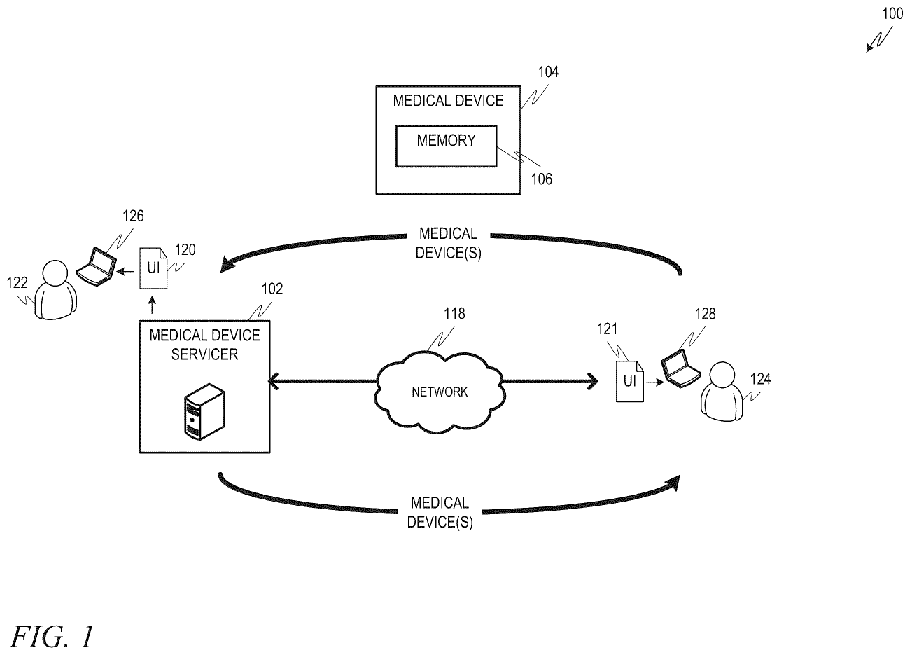

FIG. 1 is a diagram showing one example of an environment 100 for supporting medical device repair. The environment 100 includes a medical device servicer system 102. The medical device servicer system 102 may comprise one or more computing devices programmed to execute various software programs such as, for example, a medical device service program. The medical device servicer system 102 may provide a user interface 120 to one or more service users 122 via one or more user computing devices 126.

In various examples, the medical device servicer system 102 is implemented by a medical device servicer entity that services medical devices. The medical device servicer entity, in some examples, is a manufacturer who manufactures the medical devices. In other examples, the medical device servicer entity is a re-processor or other third-party entity that services medical devices. The service users 122, for example, may be users associated with the medical device servicer who service medical devices and/or manage the service of medical devices. The user interface 120, for example, may receive inputs from the one or more service users 122 and/or provide outputs to the one or more service users 122. The inputs and/or outputs may be related to the service of medical devices, as described herein.

The environment 100 also includes a medical device consumer user 124. The medical device consumer user 124 is associated with a medical device consumer. The medical device consumer user 124, via the user computing device 128, may receive a user interface 121 generated by the medical device servicer system 102 via a network 118.

The medical device consumer user 124 (and/or an entity associated with the medical device consumer user 124) may provide a medical device 104 to the medical device servicer entity. The medical device 104, for example, may be owned by the medical device consumer entity and be in need of repair or other service.

In some examples, medical devices, such as the medical device 104, may have a suggested usage life. The suggested usage life is the period of service during which the medical device 104 can be safely and effectively used to perform surgical procedures. For example, the medical device servicer entity may decline to provide service to the medical device 104 after the expiration of its suggested usage life. The medical device servicer system 102, in some examples, is programmed to execute a service management application that determines when medical devices, such as the medical device 104, are suitable for continued use, for example, after the expiration of a usage life threshold. The service management application may be accessed, for example, by the service user 122 via the user interface 120 and/or via the medical device consumer user 124 via the user interface 121.

In some examples, the medical device servicer entity may implement the service management application as part of an equipment-as-a-service (EaaS) arrangement for providing medical devices to the medical device consumer entity. For example, the medical device servicer may own and sterilize and/or reprocess medical devices, such as endoscopes, consumables for use with endoscopes such as, for example, endoscope valves, endoscope tips, and the like. In other examples, other medical devices may be used. According to an EaaS arrangement, the medical device provider owns and provides medical devices to the medical device consumer on a cost per use or cost per procedure basis. The medical device provider may arrange for sterilization and/or reprocessing of the medical devices and provide any inventory of the medical devices for use by the medical device consumer.

The medical device servicer system 102 may be directly or indirectly in communication with the user computing device 126 and/or the user computing device 128 via a network 118. The network 118 may be or include any suitable wired, wireless, or mixed network including, for example, a wide area network (WAN), a local area network (LAN) and/or the like.

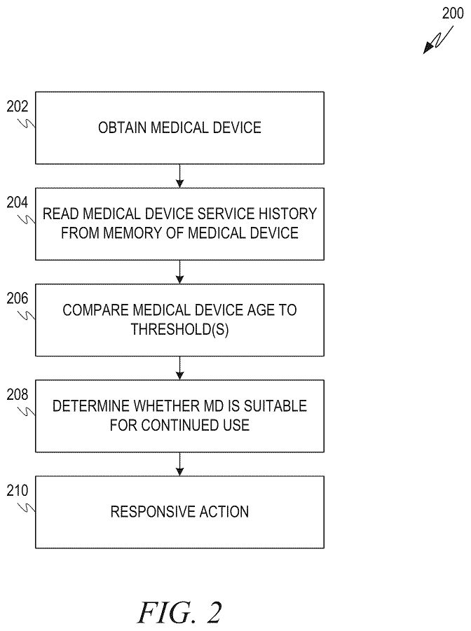

FIG. 2 is a flowchart showing one example of a process flow 200 that may be executed by the medical device servicer system 102 (e.g., the service management application) and/or the service user 122 to determine whether the medical device 104 is eligible for service. At operation 202, the service user 122 obtains the medical device 104. For example, the medical device consumer user 124 and/or another user at the medical device consumer may provide the medical device 104 to the medical device servicer for service. The requested service may include, for example, sterilization, reprocessing, repair, and/or the like.

At operation 204, the medical device servicer system 102 reads a service and/or usage history for the medical device 104 from a memory 106 associated with the medical device 104. The memory 106, for example, may be an onboard memory positioned on, in, or otherwise associated with the medical device 104. The service history may indicate a number and/or type of repairs or other services made to the medical device 104. Services may include, for example, sterilizations, component replacements, and/or other repairs.

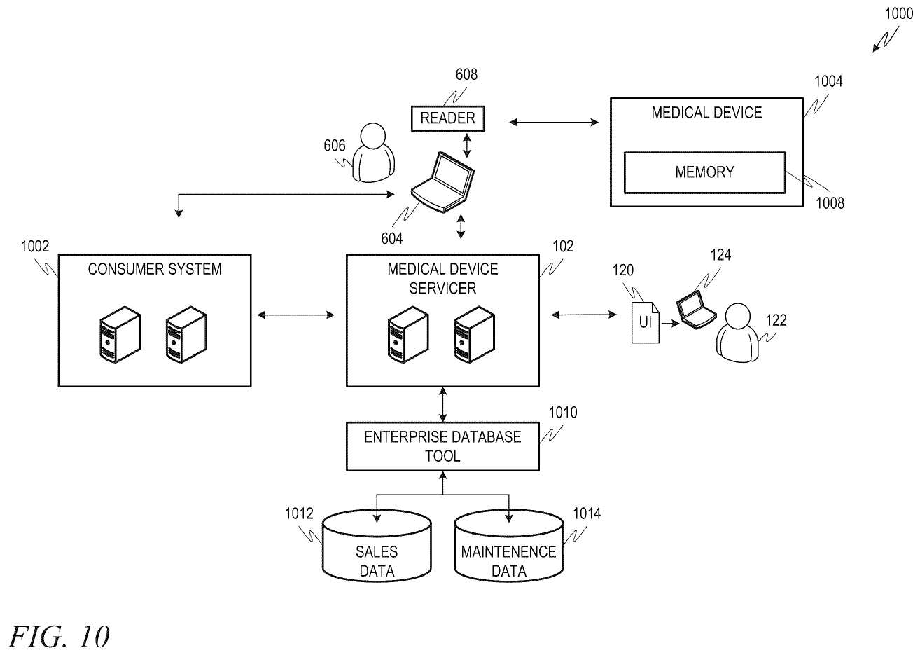

At operation 206, the medical device servicer system 102 compares an age of the medical device 104 to an age threshold. In some examples, the age of the medical device 104 is also stored at the memory 106. Also, in some examples, the medical device servicer system 102 may consult a database or other datastore indicating the age of the medical device 104, such as a datastore generated by or with data from the manufacturer of the medical device 104 (see, e.g., FIG. 10). The age threshold may indicate, for example, a suggested usage life for the medical device 104 provided, for example, by a manufacturer of the medical device 104. In other examples, the age threshold indicates a potential retirement age for the medical device 104. If the medical device 104 is below the age threshold, it may be considered suitable for continued use and/or service. The medical device servicer may perform service and return the medical device 104 to the medical device user entity.

When the medical device 104 has an age greater than the age threshold, the medical device servicer system 102 may determine, at operation 208, whether the medical device 104 is suitable for continued use at operation 208. This may include, for example, considering the repair count, repair type, equipment age, number of uses, type of uses, and/or the like. Repair count and repair type may be described by the service history data obtained at operation 204.

In some examples, repair count describes the frequency of repairs. In some examples, the repair count considered by the medical device servicer system 102 may be limited to more recent data such as, for example, the previous six months, the previous year, the previous eighteen months, and/or the like. Repair type can describe the repairs performed. In some examples, the medical device servicer system 102 categorizes repairs. In one example, repairs can be categorized as major, middle, or minor repairs. In various examples, the type of repair may be classified based on the amount of time required to perform the repair. For example, a repair requiring 15 minutes to perform may be a minor repair. On the other hand, a repair requiring several hours to perform may be a major repair. In some examples, repairs may be classified as major, middle, or minor according to predefined categories, which may be stored at a database or other data store associated with the medical device servicer system 102.

The medical device servicer system 102 may use the repair count, device age, and repair type to determine whether the medical device 104 is suitable for continued use. For example, the medical device servicer system 102 may assign a score to the medical device 104 based on the repair count, repair type, and equipment age. The medical device 104 may be considered suitable for continued use if the score meets a threshold value or range of values.

At operation 210, the medical device servicer system 102 may execute a responsive action. For example, if the medical device 104 is no longer suitable for use, the medical device servicer system 102 may send a message to the medical device consumer entity indicating that the medical device 104 is no longer suitable for use. The medical device servicer may dispose of the medical device 104 and/or return it to the medical device consumer entity. If the medical device 104 is suitable for continued use, the medical device servicer system 102 may initiate a service or repair of the medical device 104, for example, by generating an instruction to the service user 122 or another user to perform a repair. The medical device 104 may be returned to the medical device consumer entity (or another medical device consumer entity) upon repair.

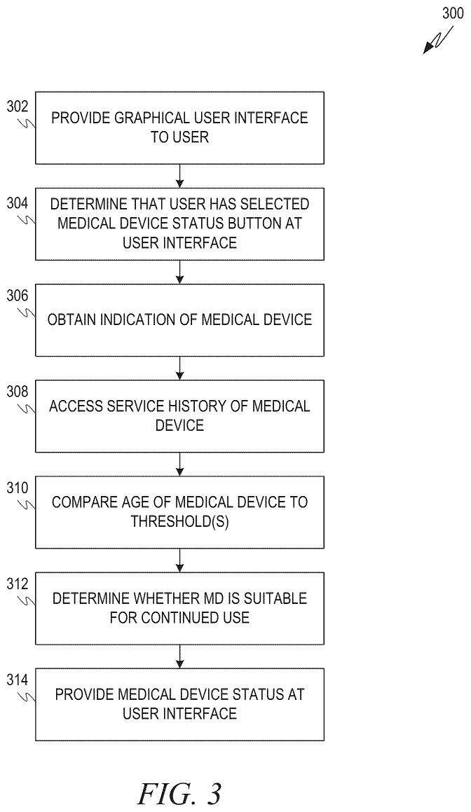

FIG. 3 is a flowchart showing one example of a process flow 300 that may be executed by the medical device servicer system to determine whether the medical device 104 is eligible for service. At operation 302, the medical device servicer system 102 provides a graphical user interface, such as the user interface 120 and/or the user interface 121. The graphical user interface may include a medical device status button selectable by the user 122, 124. At operation 304, the medical device servicer system 102 determines that the user 122, 124 has selected the medical device status button.

In response to determining that the user 122, 124 has selected the medical device status button, the medical device servicer system 102 may, at operation 306, obtain an indication of the medical device 104. For example, the user 122, 124 may input a serial number or similar identifier of the medical device 104. In other examples, the user 122, 124 may capture an image of the medical device. The image may depict a serial number or other identifier of the medical device 104 in an alphanumeric and/or coded form (e.g., bar code, QR code, etc.).

At operation 308, the medical device servicer system 102 accesses service history data and/or usage history data for the medical device 104. This service history and/or usage history data may be obtained from the memory 106 of the medical device 104 itself via an interface computing device, as described herein. For example, the user 122, 124 may connect the user computing device 126, 128 or other suitable interface computing device to the medical device 104 using a suitable wired and/or wireless communication link. The user computing device 126, 128 may read the memory 106 to determine the service history and/or usage history. Service history and/or usage history uploaded by the user computing device 126, 128 may be provided to the medical device servicer system 102.

At operation 310, the medical device servicer system 102 compares an age of the medical device 104 to an age threshold, for example, as described herein. At operation 312, the medical device servicer system 102 determines status data for the medical device 104. The status data may indicate whether the medical device 104 is suitable for continued use, for example, as described herein.

At operation 314, the medical device servicer system 102 provides the medical device status at the graphical user interface. The status may indicate whether the medical device 104 is suitable for further use. The user 122, 124 may process the medical device 104 accordingly. For example, if the medical device 104 is suitable for continued use, a service user 122 may perform a repair or other service on the medical device 104 and return it to the medical device consumer entity. A medical device consumer user 124 may make arrangements for the medical device 104 to be provided to the medical device servicer for service. If the medical device 104 is not suitable for further use, the service user 122 may dispose of the medical device 104 and/or notify the medical device consumer entity (and/or user 124) that the medical device 104 is no longer suitable for use.

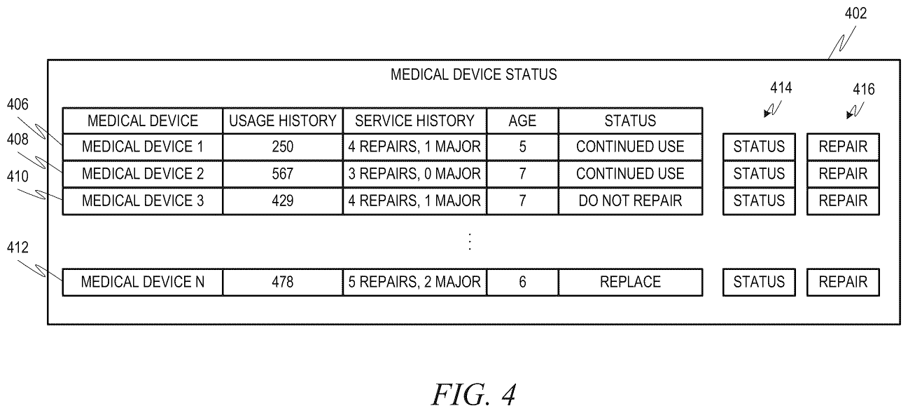

FIG. 4 is a diagram showing one example of a user interface screen 402 that may be served to a user computing device 126 associated with a medical device service user 122, for example, as part of the user interface 120. In some examples, the user interface screen 402 may be provided to a medical device consumer user 124 via user computing device 128, for example, as part of the user interface 121. The user interface screen 402 comprises records 406, 408, 410, 412. The records 406, 408, 410, 412 correspond to respective medical devices that may be utilized by the medical device consumer user 124. For example, the user interface screen 402 may display fleet audit data describing status dated generated for a plurality of medical devices.

Each record shown at the user interface screen 402 may have corresponding fields indicated by the columns MEDICAL DEVICE, USAGE HISTORY, SERVICE HISTORY, AGE, and STATUS. In the example of FIG. 4, four medical devices are described, MEDICAL DEVICE 1, MEDICAL DEVICE 2, MEDICAL DEVICE 3, and MEDICAL DEVICE N. Although four medical devices are described in FIG. 4, it will be appreciated that the user interface screen 402 may comprise data describing more or fewer medical devices.

In the example of FIG. 4, MEDICAL DEVICE 1 has usage history indicating 250 procedures, a service history indicating four repairs and one major repair, and an age of five years. In this example, MEDICAL DEVICE 1 has status data indicating that MEDICAL DEVICE 1 is suitable for continued use.

MEDICAL DEVICE 2 has usage history indicating 567 procedures, service history indicating three repairs and zero major repairs, an age of seven years, and status data indicating that MEDICAL DEVICE 2 is suitable for continued use. MEDICAL DEVICE 3 has a usage history indicating 429 procedures, service history indicating four repairs and one major repair, an age of seven years, and status data indicating that MEDICAL DEVICE 3 may be used, but should not be repaired upon a next required repair. MEDICAL DEVICE N as a usage history indicating 478 procedures, service history indicating five repairs and two major repairs, an age of six years, and status data indicating that MEDICAL DEVICE N should be replaced.

The user interface screen 402 also includes status buttons 414 and repair buttons 416. In this example, each respective medical device has an associated status buttons 414 and an associated repair buttons 416. The status buttons 414 may be selectable by the medical device consumer user 124 to prompt the medical device servicer system 102 to determine and/or redetermine status data for the corresponding medical device. Repair buttons 416 may be selectable by the medical device consumer user 124 to prompt the medical device servicer system 102 to initiate a repair procedure for the corresponding medical device. In some examples, the user interface screen 402 may comprise a universal status button (not shown in FIG. 4) that, when selected by the medical device consumer user 124, prompts the medical device servicer system 102 to determine and/or redetermine status data for all medical devices listed on the screen 402.

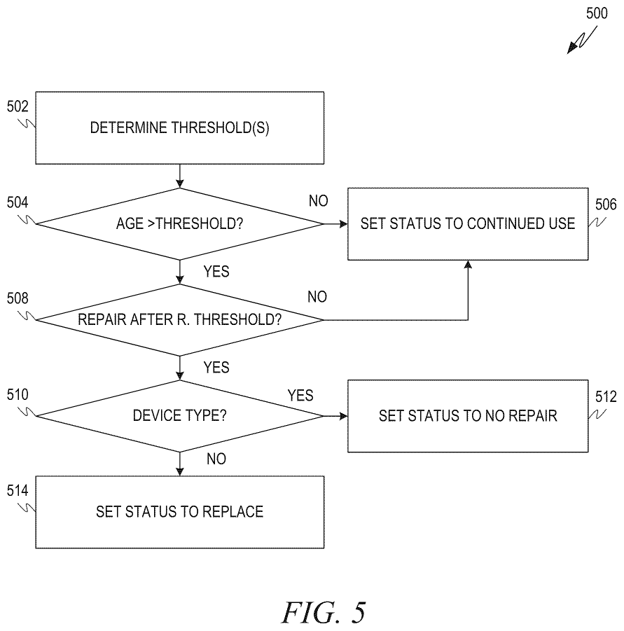

FIG. 5 is a flowchart showing one example of a process flow 500 that may be executed, for example, by the medical device servicer system 102, to determine status data for a medical device. In the example of FIG. 5, the medical device servicer system 102 determines the status data for a medical device using usage history data describing usage of the medical device, service history data describing one or more repairs to the medical device, and age data describing an age of the medical device.

At operation 502, the medical device servicer system 102 may determine one or more thresholds that will be used in the process flow 500. For example, the medical device servicer system 102 may determine an age threshold. As described herein, the age threshold may determine one or more thresholds or other rules to be applied to the medical device. The age threshold, in various examples, may be selected based on usage history data for the medical device. For example, a medical device that has been used for a higher number of procedures may be analyzed using a relatively lower age threshold. Also, for example, a medical device that has been more vigorously used may be analyzed using a relatively lower age threshold.

In some examples, the medical device servicer system 102 may also determine a repair threshold. The repair threshold may describe an age of the medical device after which the need for a major repair may indicate the end of the usage life of the medical device. In some examples, repair threshold may also be determined utilizing the usage history data. For example, a medical device that is used for a higher number of procedures may be analyzed using a relatively lower repair threshold. Also, for example, a medical device that has been more vigorously used may be analyzed using a relatively lower repair threshold. The repair threshold may be shorter than the age threshold.

At operation 504, the medical device servicer system 102 may determine, using age data for the medical device, if the age of the medical device is greater than the age threshold. If the age of the medical device is not greater than the age threshold, then the medical device servicer system 102 may set a status of the medical device to indicate that the medical device is suitable for continued use. If the age of the medical device is greater than the age threshold, then the medical device servicer system 102 may proceed to operation 508.

At operation 508, the medical device servicer system 102 may determine if the medical device has experienced a repair after the repair threshold. If the medical device has not experienced a repair after the repair threshold, then the medical device servicer system 102 may set the status of the medical device to indicate that the medical device is suitable for continued use. If the medical device has experienced a repair after the repair threshold, then the medical device servicer system 102 may proceed to operation 510.

In some examples, the repair threshold applied at operation 508 may be based on major repairs. A major repair may be determined in any suitable manner. For example, a major repair may occur when a repair procedure on the medical device takes more than a threshold amount of time, when certain components of the medical device are affected, and/or the like. When the repair threshold applied at operation 508 is based on major repairs, the medical device servicer system 102 may proceed to operation 506 when the medical device has not experienced a major repair after the repair threshold, and to operation 510 when the medical device has experienced a major repair after the repair threshold.

At operation 510, the medical device servicer system 102 may determine whether the medical device is of a specialty type having a longer suggested usage life. For example, some medical devices may have different components and/or designs that extend the suggested usage life. If the considered medical device is of a device type having a longer suggested usage life, the medical device servicer system 102 may, at operation 512, set the status of the medical device to indicate that it should not be repaired. A medical device that should not be repaired may be suitable for continued use, but may not be accepted for repair should repair be required in the future.

If, at operation 510, the medical device servicer system 102 determines that the medical device is not of a type having a longer suggested usage life, then the medical device servicer system 102 may, at operation 514, set the status of the medical device to replace. This may indicate that the medical device is no longer suitable for continued use and should be retired and, if desired, replaced.

In some examples, the medical device servicer system 102 may apply thresholds in addition to or instead of the thresholds described with respect to FIG. 5. In some examples, the medical device servicer system 102 may apply an equipment utilization threshold to a medical device. The equipment utilization threshold may be determined by the medical device servicer system 102, for example, as described herein with respect to operation 502. Equipment utilization may describe a repair rate for a medical device. The repair rate may be described, for example, as a ratio relating the number of uses of the medical device and the number of repairs for the medical device. A medical device with a higher number of uses per repair may be more likely to be suitable for further use. In some examples, some or all of the thresholds applied by the medical device servicer system 102 to determine status data for a medical device a be measured relative to other medical devices within a group of medical devices. For example, a medical device may be compared to other medical devices manufactured at the same time, and/or provided to the same medical device consumer. In some examples, one or more of the thresholds may be determined based on a distribution across the group of medical devices. For example, one or more thresholds may be set such that a predetermined portion of the group of medical devices asses the threshold and is determined to be suitable for further use.

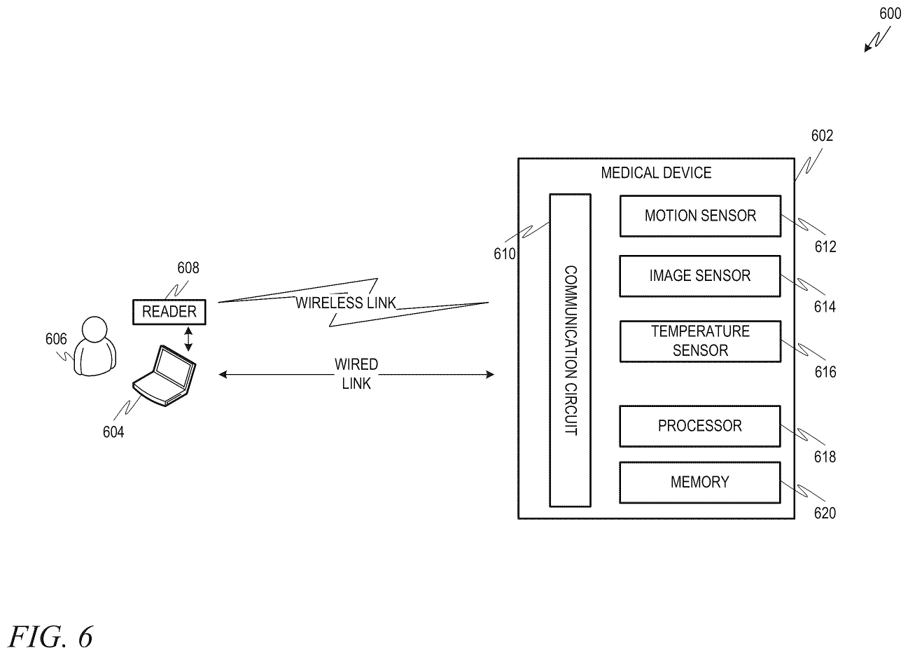

FIG. 6 is a diagram showing one example of an environment 600 including an example medical device 602 in communication with an interface computing device, such as a reader device 608 and/or a user computing device 604. The medical device 602 comprises one or more sensors 612, 614, 616 that may be used to detect usage of the medical device 602. For example, a motion sensor 612, such as an accelerometer or other suitable sensor, may detect movement of the medical device 602 indicating usage of the medical device 602 to perform a procedure. An image sensor 614, such as a camera or other suitable sensor, may capture one or more images. Images captured by the image sensor 614 may indicate use of the medical device 602 and a procedure. A temperature sensor 616 may detect changes in temperature in the environment of the medical device 602. Temperature changes in the environment of the medical device 602 determined using the temperature sensor 616 may also, in some examples, indicate that the medical device 602 has been used in a procedure. For example, when the medical device 602 is an endoscope, the temperature at a distal portion of the endoscope may rise to body temperature when the endoscope is used in a procedure. Although a motion sensor 612, image sensor, 614, and temperature sensor 616 are described in FIG. 6, it will be appreciated that various other types of sensors may be used in addition to or instead of the sensors 612, 614, 616 to detect uses of the medical device 602.

The medical device 602 also comprises a processor circuit 618 and an onboard memory 620. The processor circuit 618 may be in communication with one or more of the sensors 612, 614, 616, for example, via a suitable analog front end (not shown in FIG. 6). Processor circuit 618 may analyze signals received from the various sensors 612, 614, 616 and detect a use of the medical device 602, for example, in a procedure. When the processor circuit 618 detects a use of the medical device 602, it may store an indication of the use at the memory 620. In this way, the memory 620 may store usage history data for the medical device 602.

The medical device 602 also comprises a communication circuit 610. The communication circuit 610 may be configured to support a communication link between the medical device 602 and an interface computing device, such as the reader device 608 and/or the user computing device 604. The communication circuit 610 may be configured to establish any suitable communication link such as, for example, a wired link and/or a wireless link. In examples in which the communication circuit 610 supports a wired communication link, the communication circuit 610 may comprise a suitable socket for receiving a wire. Another end of the wire may be electrically coupled to the reader device 608 and/or the user computing device 604 to establish a wired communication link. Any suitable wired communication protocol may be used including, for example, the Universal Serial Bus (USB) protocol.

In examples in which the communication circuit 610 supports a wireless communication link, the communication circuit 610 may comprise a suitable transceiver and/or antenna for establishing the wireless communication link. Any suitable wireless communication protocol may be used including, for example, Bluetooth®, Near Field Communication (NFC), Radio Frequency Identification (RFID), and/or the like.

An interface computing device, such as the reader device 608 and/or the user computing device 604, may initiate the communication link and interrogate the medical device 602 to receive data stored at the memory 620, such as usage history data. The type of interface computing device used, in some examples, is based on the type of the communication link. For example, when an RFID wireless communication link is used, the user computing device 604 may utilize a reader device 608, which may be an RFID reader, to interrogate the medical device 602 via the communication circuit 610. It will be appreciated that the user computing device 604 may comprise onboard capabilities for communicating using various wired and/or wireless protocols. In examples in which the communication link is established based on a wired or wireless protocol supported by the user computing device 604, such as, for example Bluetooth®, NFC, USB, and/or the like, the communication link may be established directly by the user computing device 604, which may act as the interface computing device.

A user 606 may utilize the user computing device 604 and/or reader device 608 to establish the communication link with the medical device 602 and interrogate the memory 620 to receive the usage history data. In some examples, the user 606 is associated with the medical device servicer. For example, user may be a technician or other staff member who analyzes the medical device 602 when it is provided to the medical device servicer for repair or service. In other examples, the user 606 travels to a facility associated with the medical device consumer to perform a fleet audit of medical devices owned and/or operated by the medical device consumer. The user 606 may use the user computing device 604 and/or the reader device 608 at the facility associated with the medical device consumer to interrogate the medical device 602 (and, optionally, other medical devices of the medical device consumer).

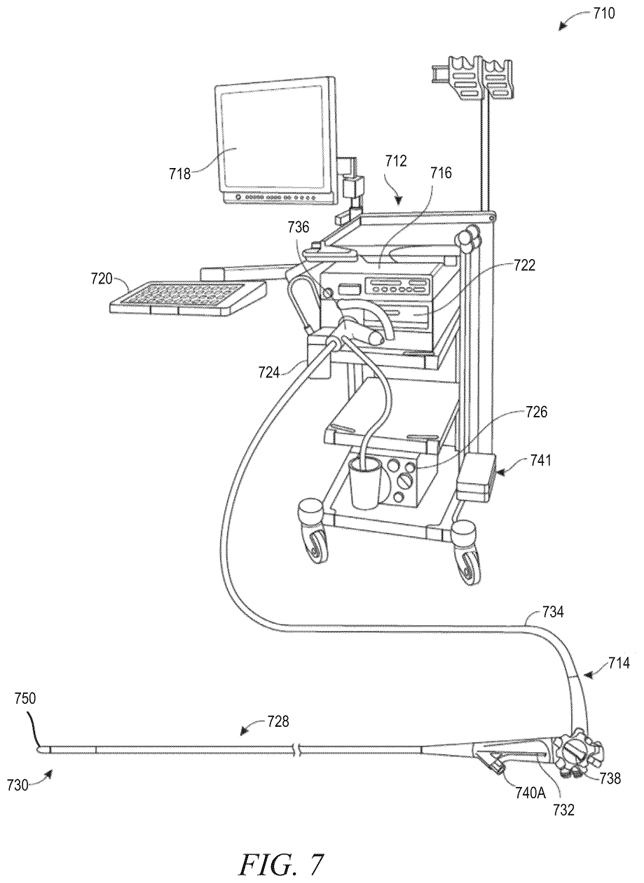

FIG. 7 is a schematic diagram of one example of an endoscopy system 710 comprising imaging and control system 712 and endoscope 714. The endoscope 714 is one example of a medical device that may be utilized in the manner described herein.

According to some examples, endoscope 714 can be insertable into an anatomical region for imaging and/or to provide passage of other devices, such as auxiliary scopes and biopsy devices or one or more therapeutic devices for treatment of a disease state associated with the anatomical region. Endoscope 714 can, in beneficial aspects, interface with and connect to imaging and control system 712. In the illustrated example, endoscope 714 comprises a duodenoscope, though other types of endoscopes can be used with the features and teachings of the present disclosure.

Imaging and control system 712 can comprise control unit 716, output unit 718, input unit 720, light source unit 722, fluid source 724, and suction pump 726.

Imaging and control system 712 can include various ports for coupling with endoscopy system 710. For example, control unit 716 can include a data input/output port for receiving data from and communicating data to endoscope 714. Light source unit 722 can include an output port for transmitting light to endoscope 714, such as via a fiber optic link. Fluid source 724 can include a port for transmitting fluid to endoscope 714. Fluid source 724 can comprise a pump and a tank of fluid or can be connected to an external tank, vessel or storage unit. Suction pump 726 can comprise a port used to draw a vacuum from endoscope 714 to generate suction, such as for withdrawing fluid from the anatomical region into which endoscope 714 is inserted. Output unit 718 and input unit 720 can be used by an operator of endoscopy system 710 to control functions of endoscopy system 710 and view output of endoscope 714.

Control unit 716 can additionally be used to generate signals or other outputs from treating the anatomical region into which endoscope 714 is inserted. In examples, control unit 716 can generate electrical output, acoustic output, a fluid output, and the like for treating the anatomical region with, for example, cauterizing, cutting, freezing, and the like.

Endoscope 714 can comprise insertion section 728, functional section 730, and handle section 732, which can be coupled to cable section 734 and coupler section 736. Coupler section 736 can be connected to control unit 716 to connect to endoscope 714 to multiple features of control unit 716, such as input unit 720, light source unit 722, fluid source 724, and suction pump 726.

Insertion section 728 can extend distally from handle section 732, and cable section 734 can extend proximally from handle section 732. Insertion section 728 can be elongate and include a bending section and a distal end to which functional section 730 can be attached. The bending section can be controllable (e.g., by control knob 738 on handle section 732) to maneuver the distal end through tortuous anatomical passageways (e.g., stomach, duodenum, kidney, ureter, etc.). Insertion section 728 can also include one or more working channels (e.g., an internal lumen) that can be elongate and support insertion of one or more therapeutic tools of functional section 730, such as an auxiliary scope. The working channel can extend between handle section 732 and functional section 730. Additional functionalities, such as fluid passages, guide wires, and pull wires can also be provided by insertion section 728 (e.g., via suction or irrigation passageways, and the like).

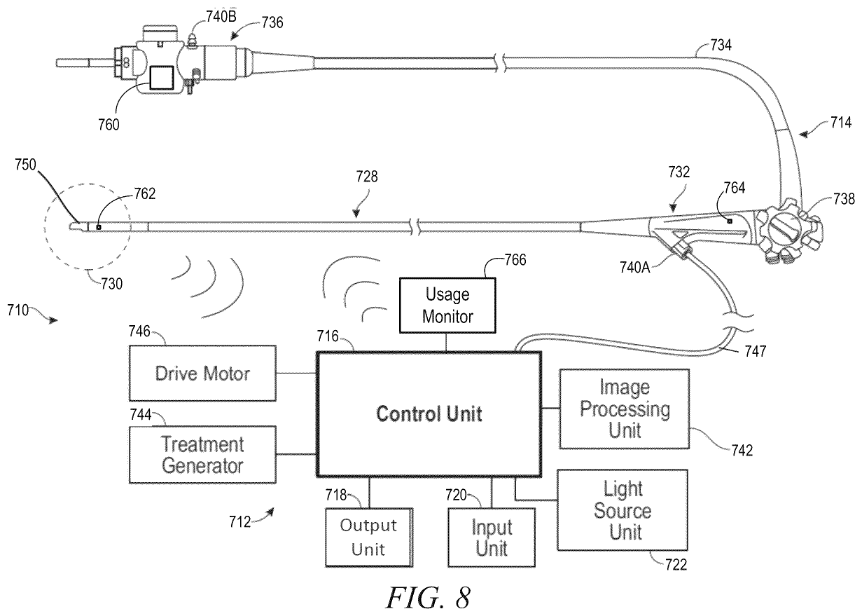

Handle section 732 can comprise control knob 738 as well as port 740A. Control knob 738 can be coupled to a pull wire, or other actuation mechanisms, extending through insertion section 728. Port 740A, as well as other ports, such as port 740B (FIG. 8), can be configured to couple various electrical cables, guide wires, auxiliary scopes, tissue collection devices, fluid tubes, and the like to handle section 732 for coupling with insertion section 728.

Imaging and control system 712, according to examples, can be provided on a mobile platform (e.g., cart 741) with shelves for housing light source unit 722, suction pump 726, image processing unit 742 (FIG. 8), etc. Alternatively, several components of imaging and control system 712 shown in FIGS. 7 and 8 can be provided directly on endoscope 714 so as to make the endoscope “self-contained.”

Functional section 730 can comprise components for treating and diagnosing anatomy of a patient, such as a distal tip 750. Functional section 730 can comprise an imaging device, an illumination device, and an elevator, for example.

FIG. 8 is a schematic diagram of one example of the endoscopy system 710 of FIG. 7 comprising imaging and control system 712 and endoscope 714. FIG. 8 schematically illustrates components of imaging and control system 712 coupled to endoscope 714, which in the illustrated example comprises a duodenoscope. Imaging and control system 712 can comprise control unit 716, which can include or be coupled to image processing unit 742, treatment generator 744, and drive unit 746, as well as light source unit 722, input unit 720, and output unit 718. Coupler section 736 can be connected to control unit 716 to connect to endoscope 714 to multiple features of control unit 716, such as image processing unit 742 and treatment generator 744. In examples, port 740A can be used to insert another instrument or device, such as a daughter scope or auxiliary scope, into endoscope 714. Such instruments and devices can be independently connected to control unit 716 via cable 747. In examples, port 740B can be used to connect coupler section 736 to various inputs and outputs, such as video, air, light, and electric. Control unit 716 can be configured to activate a camera to view target tissue distal of endoscope 714. Likewise, control unit 716 can be configured to activate light source unit 722 to shine light on endoscope 714 or other devices extending therefrom.

Image processing unit 742 and light source unit 722 can each interface with endoscope 714 (e.g., at functional section 730) by wired or wireless electrical connections. Imaging and control system 712 can accordingly illuminate an anatomical region, collect signals representing the anatomical region, process signals representing the anatomical region, and display images representing the anatomical region on output unit 718. Imaging and control system 712 can include light source unit 722 to illuminate the anatomical region using light of desired spectrum (e.g., broadband white light, narrow-band imaging using preferred electromagnetic wavelengths, and the like). Imaging and control system 712 can connect (e.g., via an endoscope connector) to endoscope 714 for signal transmission (e.g., light output from light source, video signals from imaging system in the distal end, diagnostic and sensor signals from a diagnostic device, and the like).

Fluid source 724 (FIG. 7) can be in communication with control unit 716 and can comprise one or more sources of air, saline, or other fluids, as well as associated fluid pathways (e.g., air channels, irrigation channels, suction channels) and connectors (barb fittings, fluid seals, valves, and the like). Fluid source 724 can be utilized as an activation energy for a biasing device or a pressure-applying device of the present disclosure. Imaging and control system 712 can also include drive motor 746, which can be an optional component. Drive motor 746 can comprise a motorized drive for advancing a distal section of endoscope 714, as described in at least PCT Pub. No. WO 2011/140118 A1 to Frassica et al., titled “Rotate-to-Advance Catheterization System,” which is hereby incorporated in its entirety by this reference.

FIG. 8 also illustrates an example placement of a control circuit 760 and sensors 762, 764. The control circuit 760 is positioned at a proximal portion of the endoscope 714 and may comprise a processor and an onboard memory, such as the processor circuit 618 and memory 620. In some examples, the control circuit 760 may also comprise one or more sensors such as, for example, a motion sensor, a temperature sensor, and/or the like. Sensors 762, 764 may be any suitable sensor type and may be electrically coupled to the control circuit 760 via any suitable wired or wireless connection.

In some examples, the sensor 762, positioned at the functional section 730 of the endoscope 714, is a temperature sensor. For example, when the endoscope 714 is used for a procedure, the functional section 730 may be warmed by the body heat of the patient, thereby indicating the use of the endoscope 714. Also, in some examples, the sensor 764 may be a motion sensor. For example, when a practitioner actuates the control knobs 738, it may cause motion of the handle section 732 that may be captured by a motion sensor as the sensor 764. Motion captured by a motion sensor as the sensor 764, in various examples, may indicate a use of the endoscope 714.

FIG. 8 also shows the control system 712 including a usage monitor circuit 766. The usage monitor circuit 766 may be in communication with the control circuit 760. The usage monitor circuit 766 may be configured to monitor aspects of the control system 712 to detect a usage of the endoscope 714. For example, the usage monitor circuit 766 may be configured to detect operations of the drive motor 746, treatment generator 744, image processing unit 742, light source unit 722, output unit 718, and/or input unit 720 that indicate that the endoscope 714 has been used. When the usage monitor circuit 766 detects a usage of the endoscope 714, it may send a signal indicating the usage to the control circuit 760. The control circuit 760 may store an indication of the usage at an onboard memory, such as the memory 620 and in FIG. 6.

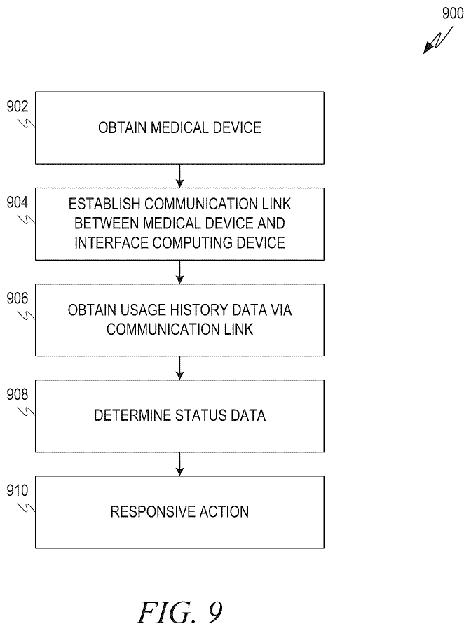

FIG. 9 is a flowchart showing one example of a process flow 900 that may be executed to determine a status of a medical device. At operation 902, a medical device may be obtained. For example, a user, such as the user 606, associated with the medical device servicer may receive a medical device at a facility of the medical device servicer and/or travel to a facility of the medical device consumer to obtain the medical device.

At operation 904, an interface computing device may establish a communication link between the interface computing device and the medical device. The communication link may be a wired and/or wireless link, as described herein. In some examples, a user, such as the user 606, may initiate a communication link using a user computing device 604 and or reader device 608 as described herein.

At operation 906, the interface computing device may obtain usage history data from a memory of the medical device via the communication link. In some examples, the interface computing device may also communicate the usage history data to a medical device servicer system 102.

At operation 908, the medical device servicer system 102 may determine status data for the medical device. This may include accessing additional data describing the medical device such as, for example, service history data and age data. In some examples, the medical device servicer system 102 may receive service history data and/or age data from an enterprise database tool, as described herein with respect to FIG. 10. At operation 910, the medical device servicer system 102 may execute a responsive action. For example, the medical device servicer system 102 may provide a user interface comprising a user interface screen, such as the user interface screen 402, to a consumer user associated with the medical device consumer where the user interface screen indicates the status of the medical device. In other examples, the medical device servicer system 102 may initiate a service activity for the medical device, decline a service activity for the medical device, and/or take other suitable responsive action.

FIG. 10 is a diagram showing one example of an environment 1000 including the medical device servicer system 102 in communication with various other components. The medical device servicer system 102 is in communication with an enterprise database tool 1010. The enterprise database tool 1010 may be in communication with various databases 1012, 1014.

The databases 1012, 1014 may comprise a sales database 1012 comprising data describing the sale of medical devices and a maintenance database 1014 comprising data describing repairs and/or other maintenance performed on medical devices. Data at the databases 1012 and 1014 may be arranged in records, with each record corresponding to a respective medical device. A record for a medical device may indicate data about the medical device and may be associated with the unique identifier of the medical device. In some examples, the unique identifier of the medical device may also be stored at the onboard memory of the medical device. In this way, medical device servicer system 102 may correlate data regarding medical devices received from the user computing device 604 and/or reader device 608 with records at the enterprise database tool 1010.

The sales database 1012, in some examples, comprises records indicating the sale date for respective medical devices. The medical device servicer system 102 may query the enterprise database tool 1010 to receive the sale date for a medical device and/or the age of the medical device. In some examples, the age of the medical device may be determined by comparing the sale date of the medical device to a current date.

The maintenance database 1014 comprises records indicating repairs for the respective medical devices. For example, the record corresponding to a particular medical device may indicate repairs that were performed on the medical device and, for example, a type of repair (e.g., major, middle, or minor).

In some examples, the medical device servicer system 102 may prompt an interface computing device, such as the user computing device 604 and/or the reader device 608, to interrogate a medical device, such as the medical device 1004, to obtain usage history data for determining a status of the medical device 1004. Medical device servicer system 102 may prompt the interface computing device directly and/or may prompt the user 606 (e.g., via the user computing device 604) to establish the communication link and interrogate the medical device 1004. The interface computing device may interrogate the medical device 1004 to retrieve usage history data stored at the memory 1008, for example, as described herein.

The medical device servicer system 102 may also query the enterprise database tool 1010 to obtain age data and services to read data for the medical device 1004. Using the usage history data, the age data, and/or the service history data, the medical device servicer system 102 may determine status data for the medical device 1004, for example, in any of the ways described herein. The medical device servicer system 102 may provide the status data to a consumer user 124 as part of the user interface 121 served to a user computing device 128 of the consumer user 124. In some examples, the medical device servicer system 102 may provide the status data to a consumer system 1002. The consumer system 1002 may be a computing system associated with the medical device consumer that owns and/or uses the medical device 1004. The status data may be provided to the consumer system 1002 individually, as part of a fleet audit describing multiple medical devices associated with the medical device consumer, as a repair recommendation, and/or the like.

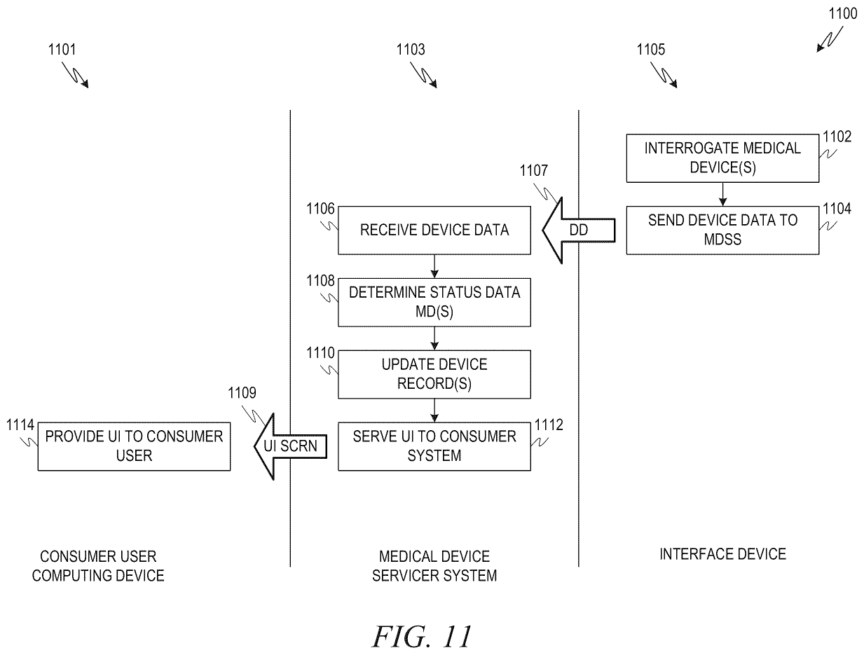

FIG. 11 is a flowchart showing one example of a process flow 1100 that may be executed in the environment 1000 to facilitate servicing of the medical device 1004. The flowchart of FIG. 11 includes three columns: 1101, 1103, and 1105. Column 1101 includes actions that may be performed a consumer user computing device 128. Column 1103 includes actions that may be performed by the medical device servicer system 102. Column 1105 includes actions that may be performed by an interface computing device, such as the user computing device 604 and/or the reader device 608.

At operation 1102, the interface computing device may interrogate a medical device or devices. This may include, for example, establishing a communication link with the medical device or devices and retrieving data from a memory of the medical device or devices such as, for example, usage history data. In some examples, the user 606 may utilize the interface computing device to interrogate multiple medical devices as a part of a fleet audit of medical devices associated with the particular medical device consumer, as a repair recommendation, and/or the like.

At operation 1104, the interface device may send retrieved medical device data 1107 to the medical device servicer system 102. The medical device servicer system 102 may receive the medical device data 1107 at operation 1106. At operation 1108, the medical device servicer system 102 may determine status data for the medical device or medical devices described by the medical device data 1107. Status data for the respective medical devices may be determined, for example, as described herein. At operation 1110, the medical device servicer system 102 may update one or more device records associated with the medical device or devices. This may include, for example, sending a request to the enterprise database tool 1010 to update records associated with the relevant medical device or devices in view of the status data determined at operation 1108.

At operation 1112, the medical device servicer system 102 may serve a user interface screen 1109 to the consumer user computing device 128. For example, the user interface screen 1109 may be part of the user interface 120. In some examples, the user interface screen 1109 is arranged in a manner similar to that of the user interface screen 402 described herein. The consumer user computing device may provide the UI screen to a consumer user at operation 1114.

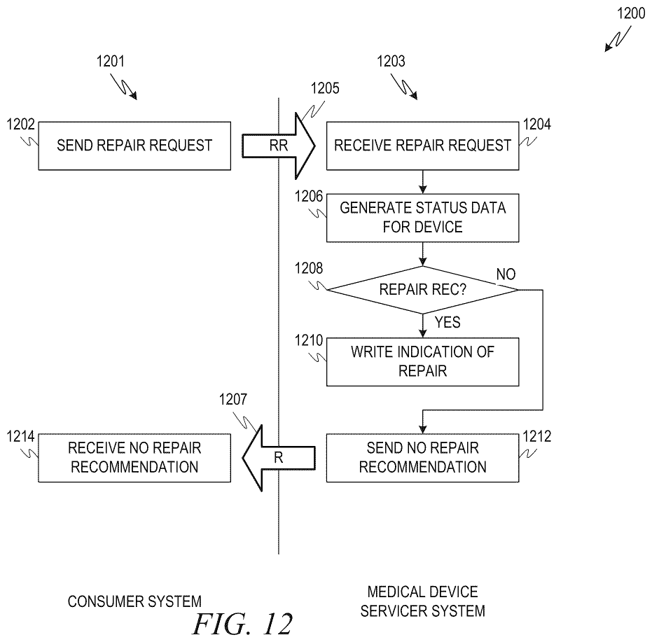

FIG. 12 is a flowchart showing another example of a process flow 1200 that may be executed, for example, in the environment 1000 of FIG. 10. FIG. 12 comprises two columns 1201, 1203. The column 1201 includes actions that may be performed by a consumer system, such as the consumer system 1002 and/or the consumer user computing device 128. The column 1203 includes actions that may be performed by the medical device servicer system 102.

At operation 1202, the consumer system may send a repair request 1205 to the medical device servicer system 102. The repair request 1205 may request the initiating of a repair service for a medical device. The medical device servicer system 102 may receive the repair request 1205 at operation 1204. At operation 1206, the medical device servicer system 102 may generate status data for the medical device indicated by the repair request 1205. This may include, for example, prompting an interface computing device to interrogate the medical device to obtain usage history data. In some examples, generating status data for the device may also include querying the enterprise database tool 1010 to obtain and/or derive service history data and age data for the medical device indicated by the repair request.

At operation 1208, the medical device servicer system 102 may determine whether repair of the indicated medical device is recommended. The determination may be made based on the status data. For example, if the status data for the medical device indicated by the repair request 1205 indicates that the medical device is suitable for continued use, then the medical device servicer system 102 may determine that repair of the medical device is recommended. In other examples, if the status data for the medical device indicates that the medical device is to be replaced, then the medical device servicer system 102 may determine that repair of the medical device is not recommended. Also, if the status data for the medical device indicates that the medical device is not to be repaired, then the medical device servicer system 102 may determine that repair of the medical device is not recommended.

If the medical device servicer system 102 determines that repair of the medical device is recommended at operation 1208, then the medical device servicer system 102 may, at operation 1210, initiate a service activity for the medical device that includes performing the requested repair. This may include, for example, generating a work order that is provided to a technician to prompt the technician to begin the repair. If the medical device servicer system 102 determines that repair of the medical device is not recommended at operation 1208, then the medical device servicer system 102 may, at operation 1212, send a no repair recommendation 1207 to the consumer system. No repair recommendation 1207 may indicate that repair of the medical device is not recommended. The consumer system may receive the no repair recommendation at operation 1214. When the medical device servicer system 102 determines that the repair of the medical device is not recommended, in some examples, the medical device servicer system 102 may not initiate a service activity to repair the medical device.

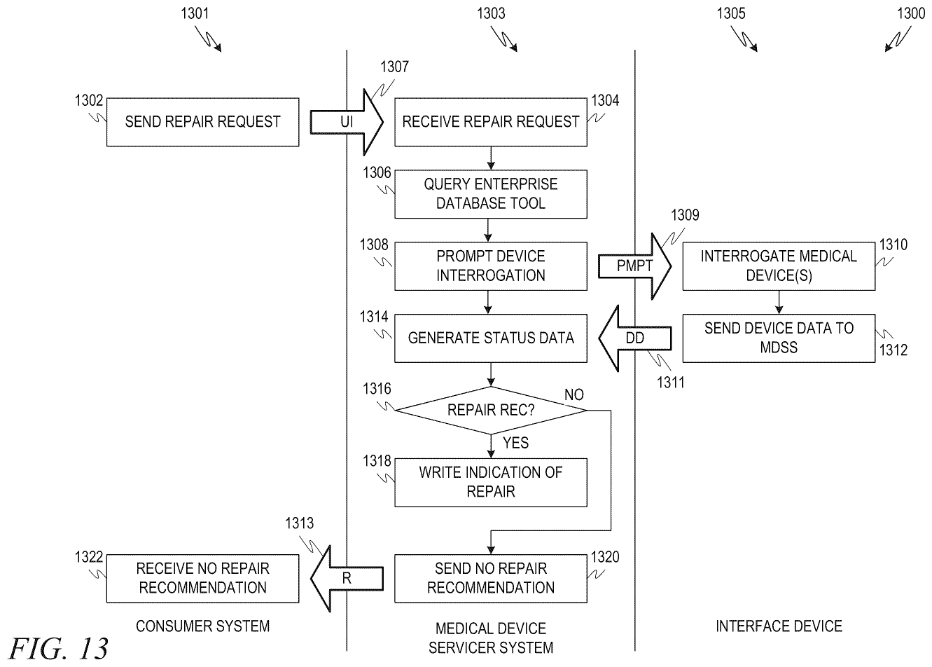

FIG. 13 is a flowchart showing another example of a process flow 1300 that may be executed, for example, in the environment 1000 of FIG. 10. FIG. 13 comprises three columns 1301, 1303, and 1305. The column 1301 includes actions that may be performed by a consumer system, such as the consumer system 1002 and/or the consumer user computing device 128. The column 1303 includes actions that may be performed by the medical device servicer system 102. The column 1305 includes actions that may be performed by an interface computing device, such as the user computing device 604 and/or the reader device 608.

At operation 1302, the consumer system may send a repair request 1307 to the medical device servicer system 102. The repair request 1307 may request the initiating of a repair service for a medical device. The medical device servicer system 102 may receive the repair request 1307 at operation 1304. At operation 1306, the medical device servicer system 102 may query the enterprise database tool 1010 to retrieve and/or derive service history data and age data for the medical device.

At operation 1308, the medical device servicer system 102 may prompt interrogation of the medical device. This may include sending a prompt 1309 to the user computing device 604 and/or reader device 608. The user computing device 604 and/or reader device 608 may respond to the prompt by interrogating the medical device. This may include establishing a communication link with the medical device and obtaining usage history data from the onboard memory of the medical device. In some examples, the user computing device 604 and/or reader device 608 may interrogate the medical device automatically upon receipt of the prompt 1309. In other examples, the prompt 1309 may cause the user computing device 604 and/or reader device 608 to provide an audio and/or visual alert to the user 606 causing the user 606 to interrogate the medical device using the user computing device 604 and/or reader device 608.

At operation 1310, the interface computing device may interrogate the medical device. At operation 1312, the interface computing device may send device data 1311 (e.g., usage history data) obtained from the onboard memory of the medical device to the medical device servicer system 102. At operation 1314, the medical device servicer system 102 may generate status data for the medical device, for example, as described herein.

At operation 1316, the medical device servicer system 102 may determine whether repair of the indicated medical device is recommended. The determination may be made based on the status data. For example, if the status data for the medical device indicated by the repair request 1307 indicates that the medical device is suitable for continued use, then the medical device servicer system 102 may determine that repair of the medical device is recommended. In other examples, if the status data for the medical device indicates that the medical device is to be replaced, then the medical device servicer system 102 may determine that repair of the medical device is not recommended. Also, if the status data for the medical device indicates that the medical device is not to be repaired, then the medical device servicer system 102 may determine that repair of the medical device is not recommended.

If the medical device servicer system 102 determines that repair of the medical device is recommended at operation 1316, then the medical device servicer system 102 may, at operation 1318, initiate a service activity for the medical device that includes performing the requested repair. This may include, for example, generating a work order that is provided to a technician to prompt the technician to begin the repair. If the medical device servicer system 102 determines that repair of the medical device is not recommended at operation 1316, then the medical device servicer system 102 may, at operation 1320, send a no repair recommendation 1313 to the consumer system. No repair recommendation 1313 may indicate that repair of the medical device is not recommended. The consumer system may receive the no repair recommendation at operation 1322. When the medical device servicer system 102 determines that the repair of the medical device is not recommended, in some examples, the medical device servicer system 102 may not initiate a service activity to repair the medical device.

### Examples

Example 1 is a method for servicing a first medical device comprising an onboard memory device, the method comprising: obtaining the first medical device; establishing, by an interface computing device, a first communication link between the first medical device and the interface computing device; obtaining first usage history data, by the interface computing device and from the onboard memory device of the first medical device via the first communication link, the first usage history data describing a number of procedures performed using the first medical device; obtaining first service history data describing at least one repair for the first medical device; obtaining first age data describing an age of the first medical device; using the first usage history data, the first age data, and the first service history data to determine first status data describing a suitability of the first medical device for continued use; and initiating, using a medical device servicer system, a service activity for the first medical device, the initiating being based at least in part on the first status data.

In Example 2, the subject matter of Example 1 optionally includes the first communication link being at least one of a wired communication link and a wireless communication link.

In Example 3, the subject matter of any one or more of Examples 1-2 optionally includes the first status data indicating that the first medical device is suitable for continued use, the method further comprising: obtaining a second medical device comprising an onboard memory device; establishing, by the interface computing device, a second communication link between the second medical device and the interface computing device; obtaining second usage history data, by the interface computing device and from the onboard memory device of the second medical device via the second communication link; obtaining second service history data describing the second medical device, the second service history data describing at least one repair for the second medical device and, for each repair, a type of repair performed; obtaining second age data describing an age of the second medical device; using the second usage history data, the second age data, and the second service history data to determine second status data indicating that the second medical device is not suitable for additional repair; and using a medical device servicer system, updating a database record associated with the second medical device to indicate the second status data.

In Example 4, the subject matter of Example 3 optionally includes accessing, by the medical device servicer system, repair request data describing a requested repair for the second medical device; and sending, by the medical device servicer system, a no repair recommendation message, the no repair recommendation message indicating that the second medical device is not to be repaired.

In Example 5, the subject matter of any one or more of Examples 3-4 optionally includes generating, by the medical device servicer system, fleet audit data describing suitability of a plurality of medical devices for continued use, the plurality of medical devices comprising the first medical device and the second medical device; and sending, by the medical device servicer system, the fleet audit data to a consumer system associated with an operator of the first medical device and the second medical device.

In Example 6, the subject matter of Example 5 optionally includes the fleet audit data comprising the first status data, the second status data, and third status data describing a third medical device, the third status data indicating that the third medical device is not suitable for continued use.

In Example 7, the subject matter of any one or more of Examples 1-6 optionally includes the determining of the first status data comprising determining, based on the first age data, that the first medical device is less than an age threshold.

In Example 8, the subject matter of any one or more of Examples 1-7 optionally includes the determining of the first status data comprising: determining, by the medical device servicer system and based on the first age data, that the first medical device is greater than an age threshold; determining, by the medical device servicer system, that a first repair of the at least one repair for the first medical device is a major repair; and determining, by the medical device servicer system, that the first repair was performed while the first medical device was less than a major repair age threshold, the first status data indicating that the first medical device is suitable for continued use.

In Example 9, the subject matter of Example 8 optionally includes determining the major repair age threshold based at least in part on the first usage history data.

In Example 10, the subject matter of any one or more of Examples 1-9 optionally includes determining, by the medical device servicer system, that the first medical device is of a first medical device type; determining, by the medical device servicer system, that a first repair of the at least one repair for the first medical device is a major repair; and determining, by the medical device servicer system, that the first repair was performed while the first medical device was greater than a major repair age threshold, the first status data indicating that the first medical device is not to be further repaired.

In Example 11, the subject matter of any one or more of Examples 1-10 optionally includes prompting, by the medical device servicer system, the interface computing device to interrogate the first medical device, the establishing of the first communication link being in response to the prompting.

In Example 12, the subject matter of any one or more of Examples 1-11 optionally includes providing, by the medical device servicer system, a graphical user interface to a user computing device, the graphical user interface comprising a medical device status button; determining, by the medical device servicer system, that a user associated with the user computing device has selected the medical device status button, the determining of the first status data being responsive to the determining that the user associated with the user computing device has selected the medical device status button; and providing, by the medical device servicer system and at the graphical user interface, an indication that the first medical device is suitable for continued use.

Example 13 is a system for servicing a first medical device comprising an onboard memory device, the method comprising: an interface computing device, the interface computing device programmed to perform operations comprising: establishing a first communication link between the first medical device and the interface computing device; and obtaining first usage history data, by the interface computing device and from the onboard memory device of the first medical device via the first communication link, the first usage history data describing a number of procedures performed using the first medical device; a medical device servicer system, the medical device servicer system programmed to perform operations comprising: obtaining first service history data describing at least one repair for the first medical device; obtaining first age data describing an age of the first medical device; using the first usage history data, the first age data, and the first service history data to determine first status data describing a suitability of the first medical device for continued use; and initiating a service activity for the first medical device, the initiating being based at least in part on the first status data.

In Example 14, the subject matter of Example 13 optionally includes the first communication link being at least one of a wired communication link and a wireless communication link.

In Example 15, the subject matter of any one or more of Examples 13-14 optionally includes the first status data indicating that the first medical device is suitable for continued use, the interface computing device being further programmed to perform operations comprising: establishing a second communication link between a second medical device comprising an onboard memory device and the interface computing device; and obtaining second usage history data, by the interface computing device and from the onboard memory device of the second medical device via the second communication link; and the medical device servicer system being further programmed to perform operations comprising: obtaining second service history data describing the second medical device, the second service history data describing at least one repair for the second medical device and, for each repair, a type of repair performed; obtaining second age data describing an age of the second medical device; using the second usage history data, the second age data, and the second service history data to determine second status data indicating that the second medical device is not suitable for additional repair; and updating a database record associated with the second medical device to indicate the second status data.

In Example 16, the subject matter of Example 15 optionally includes the medical device servicer system being further programmed to perform operations comprising: accessing repair request data describing a requested repair for the second medical device; and sending a no repair recommendation message, the no repair recommendation message indicating that the second medical device is not to be repaired.

In Example 17, the subject matter of any one or more of Examples 15-16 optionally includes the medical device servicer system being further programmed to perform operations comprising: generating fleet audit data describing suitability of a plurality of medical devices for continued use, the plurality of medical devices comprising the first medical device and the second medical device; and sending the fleet audit data to a consumer system associated with an operator of the first medical device and the second medical device.

In Example 18, the subject matter of Example 17 optionally includes the fleet audit data comprising the first status data, the second status data, and third status data describing a third medical device, the third status data indicating that the third medical device is not suitable for continued use.

In Example 19, the subject matter of any one or more of Examples 13-18 optionally includes the determining of the first status data comprising determining, based on the first age data, that the first medical device is less than an age threshold.

In Example 20, the subject matter of any one or more of Examples 13-19 optionally includes the determining of the first status data comprising: determining, based on the first age data, that the first medical device is greater than an age threshold; determining that a first repair of the at least one repair for the first medical device is a major repair; and determining that the first repair was performed while the first medical device was less than a major repair age threshold, the first status data indicating that the first medical device is suitable for continued use.

In Example 21, the subject matter of any one or more of Examples 13-20 optionally includes the medical device servicer system being further programmed to perform operations comprising determining the major repair age threshold based at least in part on the first usage history data.

In Example 22, the subject matter of any one or more of Examples 13-21 optionally includes the medical device servicer system being further programmed to perform operations comprising: determining that the first medical device is of a first medical device type; determining that a first repair of the at least one repair for the first medical device is a major repair; and determining that the first repair was performed while the first medical device was greater than a major repair age threshold, the first status data indicating that the first medical device is not to be further repaired.

In Example 23, the subject matter of any one or more of Examples 13-22 optionally includes the medical device servicer system being further programmed to perform operations comprising prompting the interface computing device to interrogate the first medical device, the establishing of the first communication link being in response to the prompting.

In Example 24, the subject matter of any one or more of Examples 13-23 optionally includes the medical device servicer system being further programmed to perform operations comprising: providing a graphical user interface to a user computing device, the graphical user interface comprising a medical device status button; determining that a user associated with the user computing device has selected the medical device status button, the determining of the first status data being responsive to the determining that the user associated with the user computing device has selected the medical device status button; and providing, at the graphical user interface, an indication that the first medical device is suitable for continued use.

Example 25 is at least one transitory machine-readable medium comprising instructions thereon that, when executed by at least one processor, cause the at least one processor to perform comprising: establishing, by an interface computing device, a first communication link between a first medical device comprising an onboard memory device and the interface computing device; obtaining, by the interface computing device, first usage history data, by the interface computing device and from the onboard memory device of the first medical device via the first communication link, the first usage history data describing a number of procedures performed using the first medical device; obtaining, by a medical device servicer system, first service history data describing at least one repair for the first medical device; obtaining, by the medical device servicer system, first age data describing an age of the first medical device; using the first usage history data, the first age data, and the first service history data to determine, by the medical device servicer system, first status data describing a suitability of the first medical device for continued use; and initiating, by the medical device servicer system, a service activity for the first medical device, the initiating being based at least in part on the first status data.

In Example 26, the subject matter of Example 25 optionally includes the first communication link being at least one of a wired communication link and a wireless communication link.

In Example 27, the subject matter of any one or more of Examples 25-26 optionally includes the first status data indicating that the first medical device is suitable for continued use, the operations further comprising: obtaining a second medical device comprising an onboard memory device; establishing, by the interface computing device, a second communication link between the second medical device and the interface computing device; obtaining second usage history data, by the interface computing device and from the onboard memory device of the second medical device via the second communication link; obtaining second service history data describing the second medical device, the second service history data describing at least one repair for the second medical device and, for each repair, a type of repair performed; obtaining second age data describing an age of the second medical device; using the second usage history data, the second age data, and the second service history data to determine second status data indicating that the second medical device is not suitable for additional repair; and using the medical device servicer system, updating a database record associated with the second medical device to indicate the second status data.

In Example 28, the subject matter of Example 27 optionally includes the operations further comprising: accessing, by the medical device servicer system, repair request data describing a requested repair for the second medical device; and sending, by the medical device servicer system, a no repair recommendation message, the no repair recommendation message indicating that the second medical device is not to be repaired.

In Example 29, the subject matter of any one or more of Examples 27-28 optionally includes the operations further comprising: generating, by the medical device servicer system, fleet audit data describing suitability of a plurality of medical devices for continued use, the plurality of medical devices comprising the first medical device and the second medical device; and sending, by the medical device servicer system, the fleet audit data to a consumer system associated with an operator of the first medical device and the second medical device.

In Example 30, the subject matter of Example 29 optionally includes the fleet audit data comprising the first status data, the second status data, and third status data describing a third medical device, the third status data indicating that the third medical device is not suitable for continued use.

In Example 31, the subject matter of any one or more of Examples 25-30 optionally includes the determining of the first status data comprising determining, based on the first age data, that the first medical device is less than an age threshold.

In Example 32, the subject matter of any one or more of Examples 25-31 optionally includes the determining of the first status data comprising: determining, by the medical device servicer system and based on the first age data, that the first medical device is greater than an age threshold; determining, by the medical device servicer system, that a first repair of the at least one repair for the first medical device is a major repair; and determining, by the medical device servicer system, that the first repair was performed while the first medical device was less than a major repair age threshold, the first status data indicating that the first medical device is suitable for continued use.

In Example 33, the subject matter of Example 32 optionally includes the operations further comprising determining the major repair age threshold based at least in part on the first usage history data.

In Example 34, the subject matter of any one or more of Examples 25-33 optionally includes the operations further comprising: determining, by the medical device servicer system, that the first medical device is of a first medical device type; determining, by the medical device servicer system, that a first repair of the at least one repair for the first medical device is a major repair; and determining, by the medical device servicer system, that the first repair was performed while the first medical device was greater than a major repair age threshold, the first status data indicating that the first medical device is not to be further repaired.

In Example 35, the subject matter of any one or more of Examples 25-34 optionally includes the operations further comprising prompting, by the medical device servicer system, the interface computing device to interrogate the first medical device, the establishing of the first communication link being in response to the prompting.

In Example 36, the subject matter of any one or more of Examples 25-35 optionally includes the operations further comprising: providing, by the medical device servicer system, a graphical user interface to a user computing device, the graphical user interface comprising a medical device status button; determining, by the medical device servicer system, that a user associated with the user computing device has selected the medical device status button, the determining of the first status data being responsive to the determining that the user associated with the user computing device has selected the medical device status button; and providing, by the medical device servicer system and at the graphical user interface, an indication that the first medical device is suitable for continued use.

Example 37 is a method for servicing a medical device comprising an onboard memory device, the method comprising: establishing, by an interface computing device, a communication link between the medical device and the interface computing device; obtaining a plurality of stored data, at least a portion of the plurality of stored data being obtained via the interface computing device and from the onboard memory device, where each of the plurality of stored data provides information specific to the medical device that influences a suitability of the medical device for continued use; and initiating, using a medical device servicer system, a service activity for the medical device, the initiating being based at least in part on the plurality of stored data.

In Example 38, the subject matter of Example 37 optionally includes the obtaining of the plurality of stored data comprising obtaining usage history data, by the interface computing device and from the onboard memory device of the medical device via the communication link, the usage history data describing a number of procedures performed using the medical device.

In Example 39, the subject matter of any one or more of Examples 37-38 optionally includes the obtaining of the plurality of stored data comprising obtaining service history data describing at least one repair for the medical device.

In Example 40, the subject matter of Example 39 optionally includes the service history data being obtained from at least one of a medical servicer system or from the medical device.

In Example 41, the subject matter of any one or more of Examples 37-40 optionally includes the obtaining of the plurality of stored data comprising obtaining age data describing an age of the medical device.

In Example 42, the subject matter of Example 41 optionally includes the age data being obtained from at least one of a medical servicer system or from the medical device.

In Example 43, the subject matter of any one or more of Examples 37-42 optionally includes the obtaining of the plurality of stored data comprising: obtaining usage history data, by the interface computing device and from the onboard memory device of the medical device via the communication link, the usage history data describing a number of procedures performed using the medical device; obtaining service history data describing at least one repair for the medical device; and obtaining age data describing an age of the medical device.

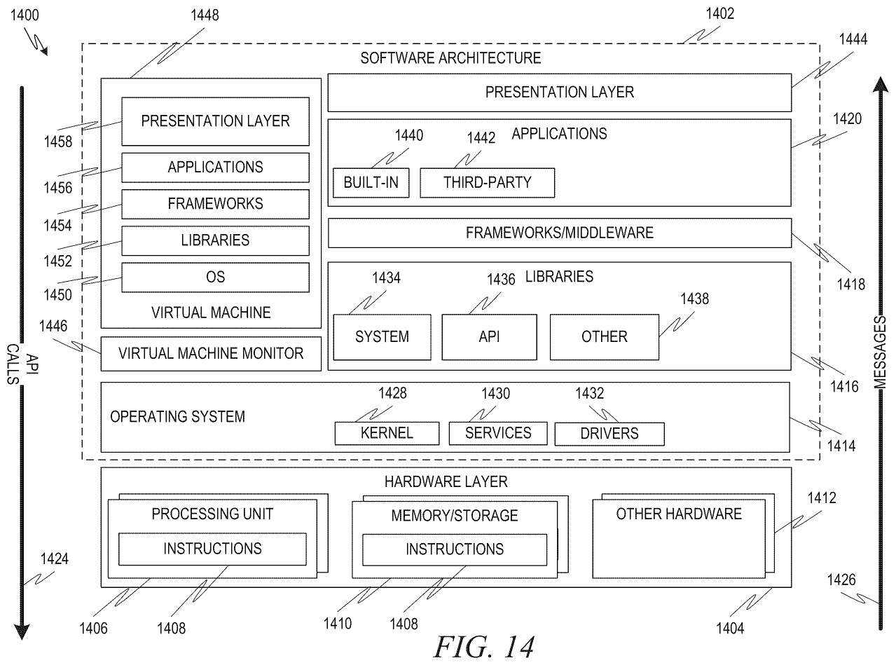

FIG. 14 is a block diagram 1400 showing one example of a software architecture 1402 for a computing device. For example, the software architecture 1402 may be used to implement all or part of the various devices described herein. The software architecture 1402 may be used in conjunction with various hardware architectures, for example, as described herein. FIG. 14 is merely a non-limiting example of a software architecture, and many other architectures may be implemented to facilitate the functionality described herein. A hardware layer 1404 is illustrated and can represent, for example, any of the above referenced computing devices. In some examples, the hardware layer 1404 may be implemented according to the architecture of the computer system of FIG. 14.

The hardware layer 1404 comprises one or more processing units 1406 having associated executable instructions 1408. Executable instructions 1408 represent the executable instructions of the software architecture 1402, including implementation of the methods, modules, subsystems, and components, and so forth described herein and may also include memory and/or storage modules 1410, which also have executable instructions 1408. Hardware layer 1404 may also comprise other hardware as indicated by other hardware 1412 which represents any other hardware of the hardware layer 1404, such as the other hardware illustrated as part of the architecture 1402.

In the example architecture of FIG. 14, the software architecture 1402 may be conceptualized as a stack of layers where each layer provides particular functionality. For example, the software architecture 1402 may include layers such as an operating system 1414, libraries 1416, middleware layer 1418, applications 1420, and presentation layer 1444. Operationally, the applications 1420 and/or other components within the layers may invoke API calls 1424 through the software stack and access a response, returned values, and so forth illustrated as messages 1426 in response to the API calls 1424. The layers illustrated are representative in nature and not all software architectures have all layers. For example, some mobile or special purpose operating systems may not provide a middleware layer 1418, while others may provide such a layer. Other software architectures may include additional or different layers.

The operating system 1414 may manage hardware resources and provide common services. The operating system 1414 may include, for example, a kernel 1428, services 1430, and drivers 1432. The kernel 1428 may act as an abstraction layer between the hardware and the other software layers. For example, the kernel 1428 may be responsible for memory management, processor management (e.g., scheduling), component management, networking, security settings, and so on. The services 1430 may provide other common services for the other software layers. In some examples, the services 1430 include an interrupt service. The interrupt service may detect the receipt of an interrupt and, in response, cause the architecture 1402 to pause its current processing and execute an interrupt service routine (ISR) when an interrupt is accessed.

The drivers 1432 may be responsible for controlling or interfacing with the underlying hardware. For instance, the drivers 1432 may include display drivers, camera drivers, Bluetooth® drivers, flash memory drivers, serial communication drivers (e.g., Universal Serial Bus (USB) drivers), Wi-Fi® drivers, NFC drivers, audio drivers, power management drivers, and so forth depending on the hardware configuration.

The libraries 1416 may provide a common infrastructure that may be utilized by the applications 1420 and/or other components and/or layers. The libraries 1416 typically provide functionality that allows other software modules to perform tasks in an easier fashion than to interface directly with the underlying operating system 1414 functionality (e.g., kernel 1428, services 1430 and/or drivers 1432). The libraries 1416 may include system 1434 libraries (e.g., C standard library) that may provide functions such as memory allocation functions, string manipulation functions, mathematic functions, and the like. In addition, the libraries 1416 may include API libraries 1436 such as media libraries (e.g., libraries to support presentation and manipulation of various media format such as MPEG4, H.264, MP3, AAC, AMR, JPG, PNG), graphics libraries (e.g., an OpenGL framework that may be used to render 2D and 3D in a graphic content on a display), database libraries (e.g., SQLite that may provide various relational database functions), web libraries (e.g., WebKit that may provide web browsing functionality), and the like. The libraries 1416 may also include a wide variety of other libraries 1438 to provide many other APIs to the applications 1420 and other software components/modules.

The middleware layer 1418 (also sometimes referred to as frameworks) may provide a higher-level common infrastructure that may be utilized by the applications 1420 and/or other software components/modules. For example, the middleware layer 1418 may provide various graphic user interface (GUI) functions, high-level resource management, high-level location services, and so forth. The middleware layer 1418 may provide a broad spectrum of other APIs that may be utilized by the applications 1420 and/or other software components/modules, some of which may be specific to a particular operating system or platform.

The applications 1420 includes built-in applications 1440 and/or third-party applications 1442. Examples of representative built-in applications 1440 may include, but are not limited to, a contacts application, a browser application, a book reader application, a location application, a media application, a messaging application, and/or a game application. Third-party applications 1442 may include any of the built-in applications 1440 as well as a broad assortment of other applications. In a specific example, the third-party application 1442 (e.g., an application developed using the Android™ or iOS™ software development kit (SDK) by an entity other than the vendor of the particular platform) may be mobile software running on a mobile operating system such as iOS™, Android™, Windows® Phone, or other mobile computing device operating systems. In this example, the third-party application 1442 may invoke the API calls 1424 provided by the mobile operating system such as operating system 1414 to facilitate functionality described herein.

The applications 1420 may utilize built-in operating system functions (e.g., kernel 1428, services 1430 and/or drivers 1432), libraries (e.g., system 1434, API libraries 1436, and other libraries 1438), and middleware layer 1418 to create user interfaces to interact with users of the system. Alternatively, or additionally, in some systems interactions with a user may occur through a presentation layer, such as presentation layer 1444. In these systems, the application/module “logic” can be separated from the aspects of the application/module that interact with a user.

Some software architectures utilize virtual machines. In the example of FIG. 14, this is illustrated by virtual machine 1448. A virtual machine creates a software environment where applications/modules can execute as if they were executing on a hardware computing device. A virtual machine is hosted by a host operating system (operating system 1414) and typically, although not always, has a virtual machine monitor 1446, which manages the operation of the virtual machine 1448 as well as the interface with the host operating system (i.e., operating system 1414). A software architecture executes within the virtual machine 1448 such as an operating system 1450, libraries 1452, frameworks/middleware 1454, applications 1456 and/or presentation layer 1458. These layers of software architecture executing within the virtual machine 1448 can be the same as corresponding layers previously described or may be different.

Certain embodiments are described herein as including logic or a number of components, modules, or mechanisms. Modules may constitute either software modules (e.g., code embodied (1) on a non-transitory machine-readable medium or (2) in a transmission signal) or hardware-implemented modules. A hardware-implemented module is a tangible unit capable of performing certain operations and may be configured or arranged in a certain manner. In example embodiments, one or more computer systems (e.g., a standalone, client, or server computer system) or one or more hardware processors may be configured by software (e.g., an application or application portion) as a hardware-implemented module that operates to perform certain operations as described herein.

In various embodiments, a hardware-implemented module may be implemented mechanically or electronically. For example, a hardware-implemented module may comprise dedicated circuitry or logic that is permanently configured (e.g., as a special-purpose processor, such as a field programmable gate array (FPGA) or an application-specific integrated circuit (ASIC)) to perform certain operations. A hardware-implemented module may also comprise programmable logic or circuitry (e.g., as encompassed within a general-purpose processor or another programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) may be driven by cost and time considerations.

Accordingly, the term “hardware-implemented module” should be understood to encompass a tangible entity, be that an entity that is physically constructed, permanently configured (e.g., hardwired), or temporarily or transitorily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses that connect the hardware-implemented modules). In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and store the output of that operation in a memory device to which it is communicatively coupled. A further hardware-implemented module may then, at a later time, access the memory device to retrieve and process the stored output. Hardware-implemented modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment, or a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., APIs).

Example embodiments may be implemented in digital electronic circuitry, or in computer hardware, firmware, or software, or in combinations of them. Example embodiments may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry, e.g., an FPGA or an ASIC.

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or in a combination of permanently and temporarily configured hardware may be a design choice. Below are set out hardware (e.g., machine) and software architectures that may be deployed, in various example embodiments.

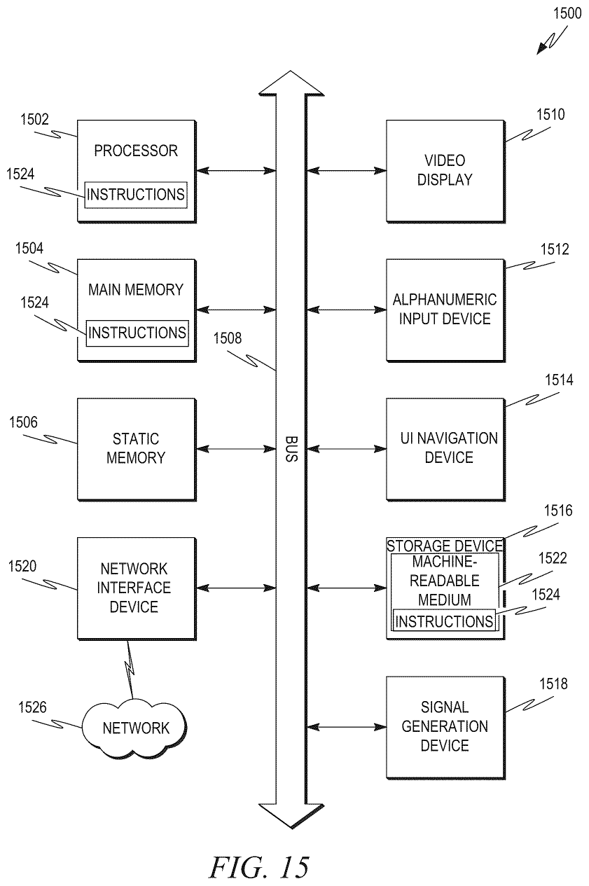

FIG. 15 is a block diagram of a machine in the example form of a computer system 1500 within which instructions 1524 may be executed for causing the machine to perform any one or more of the methodologies discussed herein. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1500 includes a processor 1502 (e.g., a central processing unit (CPU), a graphics processing unit (GPU), or both), a main memory 1504, and a static memory 1506, which communicate with each other via a bus 1508. The computer system 1500 may further include a video display unit 1510 (e.g., a liquid crystal display (LCD) or a cathode ray tube (CRT)). The computer system 1500 also includes an alphanumeric input device 1512 (e.g., a keyboard or a touch-sensitive display screen), a user interface (UI) navigation (or cursor control) device 1514 (e.g., a mouse), a disk drive unit 1516, a signal generation device 1518 (e.g., a speaker), and a network interface device 1520.

The disk drive unit 1516 includes a machine-readable medium 1522 on which is stored one or more sets of data structures and instructions 1524 (e.g., software) embodying or utilized by any one or more of the methodologies or functions described herein. The instructions 1524 may also reside, completely or at least partially, within the main memory 1504 and/or within the processor 1502 during execution thereof by the computer system 1500, with the main memory 1504 and the processor 1502 also constituting machine-readable media 1522.

While the machine-readable medium 1522 is shown in an example embodiment to be a single medium, the term “machine-readable medium” may include a single medium or multiple media (e.g., a centralized or distributed database, and/or associated caches and servers) that store the one or more instructions 1524 or data structures. The term “machine-readable medium” shall also be taken to include any tangible medium that is capable of storing, encoding, or carrying instructions 1524 for execution by the machine and that cause the machine to perform any one or more of the methodologies of the present disclosure, or that is capable of storing, encoding, or carrying data structures utilized by or associated with such instructions 1524. The term “machine-readable medium” shall accordingly be taken to include, but not be limited to, solid-state memories, and optical and magnetic media. Specific examples of machine-readable media 1522 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 1524 may further be transmitted or received over a communications network 1526 using a transmission medium. The instructions 1524 may be transmitted using the network interface device 1520 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., WiFi and WiMax networks). The term “transmission medium” shall be taken to include any intangible medium that is capable of storing, encoding, or carrying instructions 1524 for execution by the machine, and includes digital or analog communications signals or other intangible media to facilitate communication of such software.

Although an embodiment has been described with reference to specific example embodiments, it will be evident that various modifications and changes may be made to these embodiments without departing from the broader scope of the disclosure. Accordingly, the specification and drawings are to be regarded in an illustrative rather than a restrictive sense. The accompanying drawings that form a part hereof show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for servicing a first medical device comprising an onboard memory device, the method comprising:
obtaining the first medical device;
establishing, by an interface computing device, a first communication link between the first medical device and the interface computing device;
obtaining first usage history data, by the interface computing device and from the onboard memory device of the first medical device via the first communication link, the first usage history data describing a number of procedures performed using the first medical device;
obtaining first service history data describing at least one repair for the first medical device;
obtaining first age data describing an age of the first medical device;
using the first usage history data, the first age data, and the first service history data to determine first status data describing a suitability of the first medical device for continued use; and
initiating, using a medical device servicer system, a service activity for the first medical device, the initiating being based at least in part on the first status data.
